(19) United States
(12) Patent Application Publication
(10) Pub. No.: US 2015/0202086 A1
(43) Pub. Date: Jul. 23, 2015

(54) Goggle Assembly

(71) Applicant: Wang-Lee Tzu-Feng, Tainan City (TW)

(72) Inventor: Wang-Lee Tzu-Feng, Tainan City (TW)

(21) Appl. No.: 14/160,619

(22) Filed: Jan. 22, 2014

(51) Int. Cl. A61F 9/02

(52) U.S. Cl. CPC A61F 9/02

(57) ABSTRACT

A goggle assembly, wherein a spectacle frame body combined with spectacle lenses, which have a predetermined function, is formed by continuing connecting portions between an upper section and a lower section of a rigid frame body, which is near an outer periphery, with at least one connecting part to form into a single part without a break. Therefore, positions of the rigid frame body near the outer periphery don't need to be adjusted further when the rigid frame body is put into a mold. The upper section and the lower section of the rigid frame body and the connecting parts at the connecting portions are covered by injection molding, and a flexible leaning part is integrally formed near an inner periphery. Thereby, the formation of the spectacle frame body is more convenient. The spectacle frame body has an aligned exact shape and maintains its original comfort and softness.

### BACKGROUND OF THE INVENTION

1. Technical Field

The present invention relates to a goggle assembly, wherein a spectacle frame body combined with spectacle lenses, which have a predetermined function, is formed by continuing connecting portions between an upper section and a lower section of a rigid frame body, which is near an outer periphery, with at least one connecting part to form into a single part without a break. Therefore, positions of the rigid frame body near the outer periphery don't need to be adjusted further when the rigid frame body is put into a mold. The upper section and the lower section of the rigid frame body and the connecting parts at the connecting portions are covered by injection molding, and a flexible leaning part is integrally formed near an inner periphery. Thereby, the formation of the spectacle frame body is more convenient. The spectacle frame body has an aligned exact shape and maintains its original comfort and softness.

2. Description of Related Art

According to different applications, glasses assemblies made by the current industries have a variety of functional glasses, which comprise: optical glasses for vision correction, protection glasses for sports, masking glasses for protecting eyes at work, sunglasses for blocking over-strong ambient lights, and the like. Herein, protective goggles for protecting one's eyes are used for illustration purposes. The conventional goggle assembly usually comprises: a spectacle frame body, which leans around the eyes, spectacle lenses, which have a predetermined function and are combined within the spectacle frame body, and an earpiece/binding strap, assembled at two outer sides of the spectacle frame body. The spectacle frame body is formed with a rigid frame body near an outer periphery and a flexible leaning part near an inner periphery. The spectacle frame body is formed by putting the two pieces, which are an upper section and a lower section of a rigid frame body with a predetermined shape into a mold first, then two frame connecting portions between, the upper section and the lower section are covered by injection molding, and a flexible leaning part is integrally formed near the inner periphery. Then, the spectacle frame body with a desired shape and a proper flexible property at the front side is provided.

The above spectacle frame body is formed by putting the two pieces, which are the upper section and the lower section of the rigid frame body, with the predetermined shape into the mold. Though the leaning part on the inner side can be covered as expected, the two pieces of the rigid frame body are needed to be positioned in the proper position and adjusted such that the follow-up the leaning part on the inner side can be adjacently formed and aligned in the exact position. This conventional way makes the formation of the spectacle frame body comparatively more time consuming and inconvenient.

The present invention is to further improve the inconvenient problems of the formation of the abovementioned spectacle frame body and the rigid frame body on the outer periphery of the spectacle frame body is formed by continuing connecting portions between an upper section and a lower section of the rigid frame body, which is near an outer periphery, with at least one connecting part to form into a single part without a break. Therefore, positions of the rigid frame body near the outer periphery don't need to be adjusted further when the rigid frame body is put into a mold. The upper section and the lower section of the rigid frame body and the connecting parts at the connecting portions are covered by injection molding, and a flexible leaning part is integrally formed near an inner periphery. Thereby, the formation of the spectacle frame body is more convenient. The spectacle frame body has an aligned exact shape and maintains its original comfort and softness.

### BRIEF SUMMARY OF THE INVENTION

The present invention is provided to further improve the inconvenient problems of the formation of the abovementioned spectacle frame body. The spectacle frame body is formed by continuing connecting portions between an upper section and a lower section of a rigid frame body, which is near an outer periphery, with at least one connecting part to form into a single part without a break. Therefore, the rigid frame body is positioned fast and is integrated with a flexible leaning part near an inner periphery by injection molding. Thereby, the formation of the spectacle frame body is more convenient. The spectacle frame body has an aligned exact shape and maintains its original comfort and softness.

The main objective of the present invention is that the spectacle frame body of the goggle assembly is formed by continuing connecting portions between an upper section and a lower section of a rigid frame body, which is near an outer periphery, with at least one connecting part to form into a single part without a break. Therefore, positions of the rigid frame body near the outer periphery don't need to be adjusted further when the rigid frame body is put into a mold. The upper section and the lower section of the rigid frame body and the connecting parts at the connecting portions are covered by injection molding, and a flexible leaning part is integrally formed near an inner periphery. Thereby, the formation of the spectacle frame body is more convenient. The spectacle frame body has an aligned exact shape and maintains its original comfort and softness.

The second objective of the present invention is that the connecting portions between the upper section and the lower section of the rigid frame body are combined with the connecting parts, which use a material with an appropriate robustness.

### BRIEF DESCRIPTION OF THE SEVERAL VIEWS OF THE DRAWINGS

FIG. 1 is an external view of the rigid frame body of the present invention.

FIG 1A is an enlarged detail drawing of part A of FIG. 1.

FIG. 2 is an external view of a rigid frame body, which is integrally formed with the flexible leaning part, of the present invention.

FIG. 2A is an enlarged detail drawing of part A of FIG. 2.

FIG. 3 is an exploded view of the spectacle frame body and the related components of the goggle assembly of the present invention.

FIG. 4 is an external view of the assembled goggle assembly of the present invention.

### DETAILED DESCRIPTION OF THE INVENTION

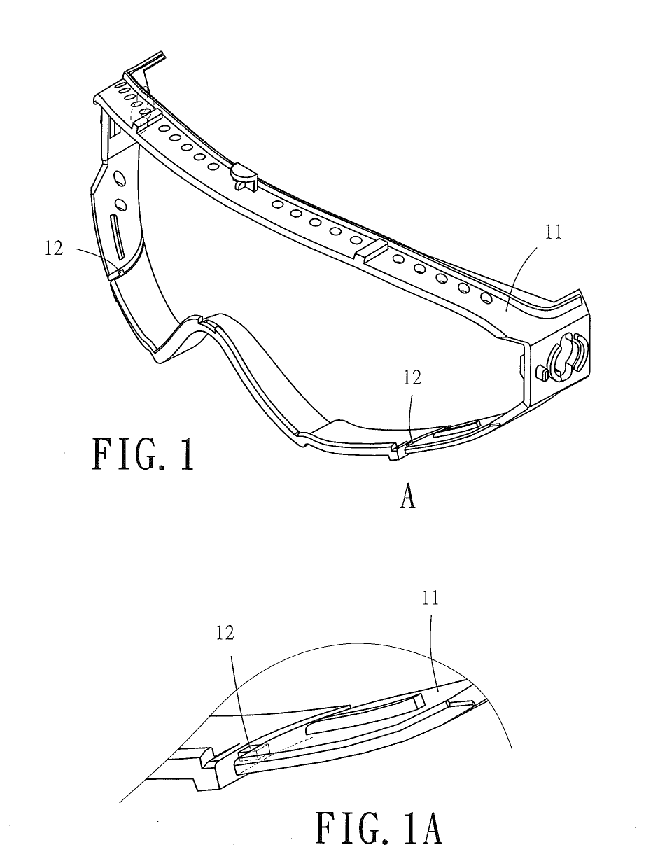

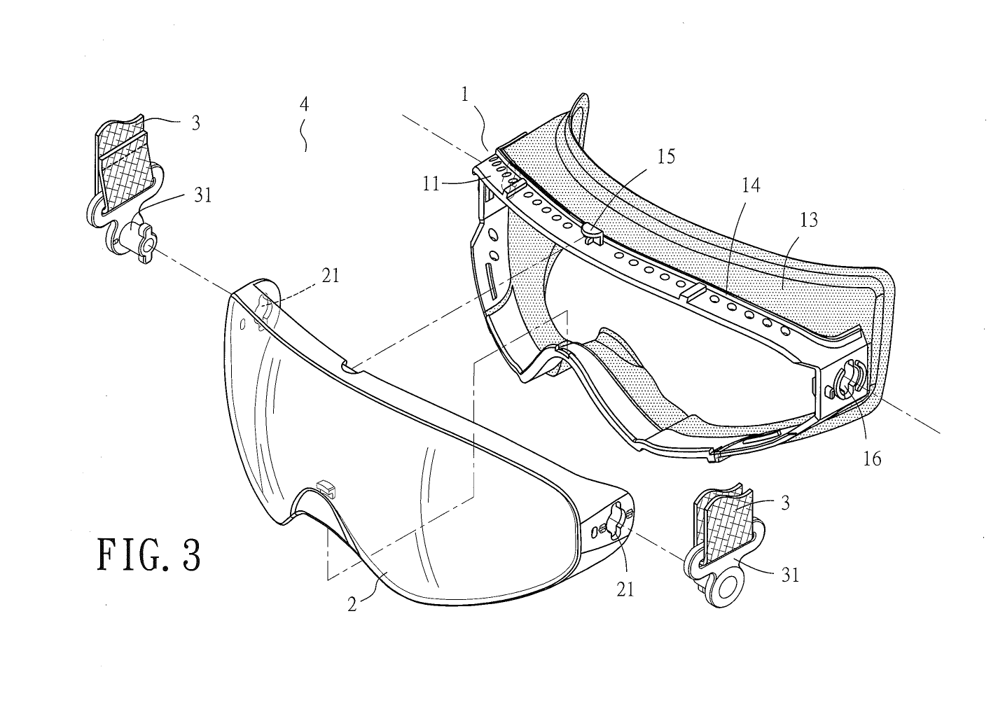

The goggle assembly designed by the present invention is shown in FIG. 3, and the goggle assembly 4 comprises: a spectacle frame body 1, spectacle lenses 2, which have a predetermined function and are combined within the spectacle frame body 1, and an earpiece/binding strap 3, assembled on two outer sides of the spectacle frame body 1. The spectacle frame body 1 has rigid frame body 11, which is near an outer periphery, and a flexible leaning part 13, which is formed near an inner periphery. The present invention improves the formation of the spectacle frame body 1. The spectacle frame body 1 is formed by continuing connecting portions between an upper section and a lower section of the rigid frame body 11, which is near the outer periphery, with at least one or more connecting part 12 to form into a single part without a break as shown in FIG. 1, FIG. 1A. The connecting portions between the upper section and the lower section of the rigid frame body 11 are combined with the connecting parts 12, which use a material with an appropriate robustness.

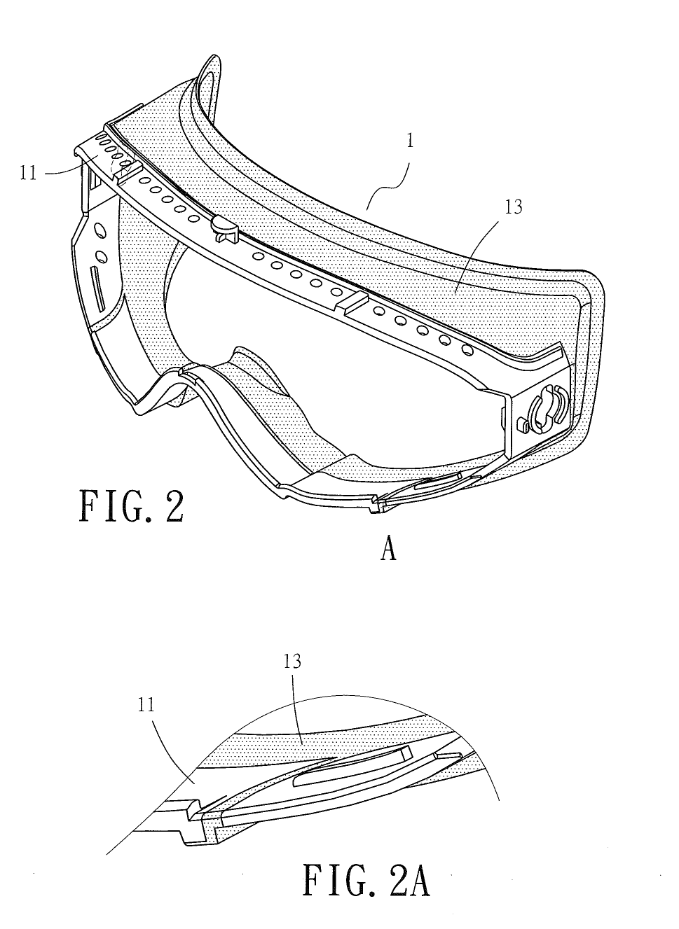

The rigid frame body 11, which is near the outer periphery, and a flexible leaning part 13, which is near the outer periphery, are implemented by putting the rigid frame body 11, which is near the outer periphery, into a preformed mold cavity, covering the upper section and the lower section of the rigid frame body 11 and the connecting parts 12 at the connecting portions by injection molding, and integrally forming the flexible leaning part 13 near the inner periphery to form the spectacle frame body 1 as shown in FIG. 2, FIG. 2A. Since the rigid frame body 11 is continued, the positions of the rigid frame body 11 near the outer periphery don't need to be adjusted further. Thereby, the formation of the spectacle frame body 1 is more convenient. The spectacle frame body has an aligned exact shape and maintains its original comfort and softness.

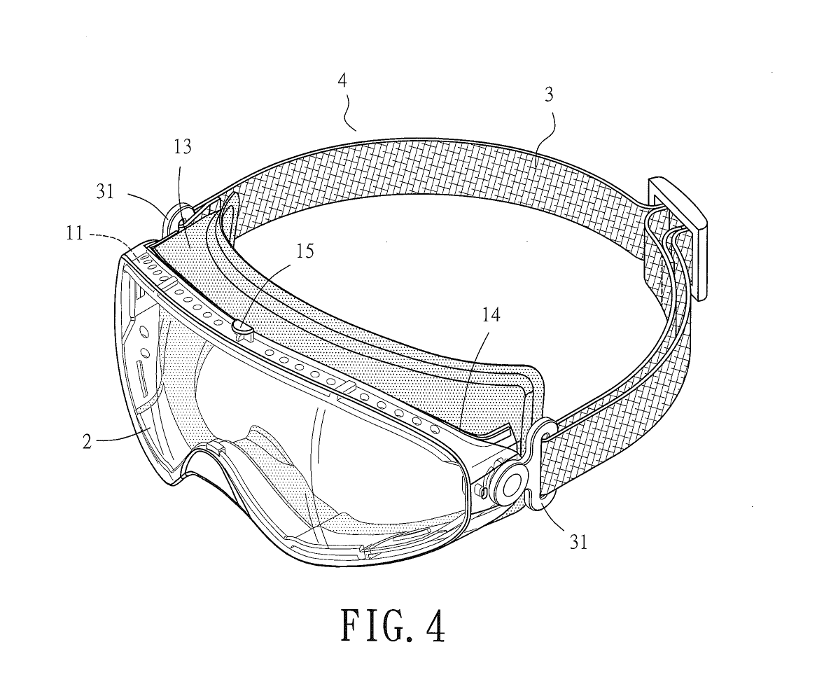

The aforementioned spectacle frame body 1 is positioned and assembled with other related components, i.e. the spectacle lenses 2 having a predetermined function and the earpiece/binding strap 3, of the goggle assembly 4 as shown in FIG. 3. The spectacle lenses 2 are correspondingly pushed to the front side of the outer periphery of the spectacle frame body 1 and the positions thereof are restricted by projecting portions 14, 15 at the outer periphery of the spectacle frame body 1. Then, fasteners 31 combined at two ends of the binding strap 3 sequentially pass through through holes 21, 16 on two outer sides of the spectacle lenses 2 and the spectacle frame body 1 in turn. The fasteners 31 at the two sides are pushed downward as shown in FIG. 4. Thereby, the binding strap 3 is fastened at the two outer sides of the spectacle lenses 2 and the spectacle frame body 1 to form the goggle assembly 4.

What is claimed is:

1. A goggle assembly, comprising a spectacle frame body, spectacle lenses, which have a predetermined function and are combined within the spectacle frame body, and an earpiece/binding strap, assembled at two outer sides of the spectacle frame body;
the improvement comprises: the spectacle frame body being formed by continuing connecting portions between an upper section and a lower section of rigid frame body, which is near an outer periphery, with at least one connecting part to form into a single part without a break;
wherein positions of the rigid frame body near the outer periphery don't need to be adjusted further when the rigid frame body is put into a mold, the upper section and the lower section of the rigid frame body t and the connecting parts at the connecting portions are covered by injection molding, a flexible leaning part is integrally formed near an inner periphery, whereby, the formation of the spectacle frame body is more convenient, and the spectacle frame body has an aligned exact shape and maintains its original comfort and softness.

2. The goggle assembly in claim 1, wherein the connecting portions between the upper section and the lower section of the rigid frame body are combined with the connecting parts, which use a material with an appropriate robustness.
